(19) United States
(12) Patent Application Publication
(10) Pub. No.: US 2003/0215400 A1
(43) Pub. Date: Nov. 20, 2003

(54) Pressurized package made of a polyamide resin and containing dimethyl ether

(75) Inventors: Kenneth Michael Schroeder, Erlanger, KY (US); Scott Edward Smith, Cincinnati, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/146,697

(22) Filed: May 15, 2002

(51) Int. Cl. A61L 9/04; A61K 9/00

(52) U.S. Cl. 424/47; 424/45

(57) ABSTRACT

A pressurized package capable of containing dimethyl ether and dimethyl ether based products such as antiperspirants and deodorants. The package may be made substantially of a polyamide.

### TECHNICAL FIELD

The present invention relates to a plastic pressurized package capable of containing dimethyl ether and dimethyl ether based products such as antiperspirants and deodorants.

### BACKGROUND OF THE INVENTION

The consumer products industry provides the world's consumers with a wide variety of products that are designed to meet consumer's needs. These personal care products are designed to not only meet the functional needs of consumers but also create a usage experience that is pleasurable. The number and variety of products that are available to today's consumers is vast and spans a broad range of functional design, aesthetic design, and intended use. These products can be grouped in numerous ways. For example, products can be grouped by function (cleansing, prevention, treatment, cosmetic enhancement, sensory experience, etc.), form (sprays, creams, lotions, wipes, bars, lathering soaps, etc.), and/or intended use (for hair, teeth, facial skin, legs, underarms, whole body). When considering the function, form and intended use, it is important to consider the package needed. Packages can be made of many materials such as plastic, glass or metal. Understanding the consumer desires, technical stability and mechanical robustness of the packaging material is necessary prior to expanding a product into the marketplace. Additional testing requirements will further drive the packaging material of choice.

In response to consumer preferences, some consumer products are being sold in plastic packages rather than glass packages. For instance, glass bottles are being replaced with polyester plastic bottles for both soda and beer. Such polyester packages may be made of polyethylene teraphthalate [PET] and/or polyethylene naphthylate [PEN]. plastics or blends or layers thereof. These polyester materials provide good containment of the aqueous portions of these consumer products, however, they do not provide good containment of the compressed gaseous fractions of these consumer products (e.g., carbon dioxide, oxygen, nitrogen). Although the compressed gaseous fractions permeate through the package, they do not substantially damage the package. However, because the permeation of the compressed gaseous fraction negatively impacts the aqueous portion still remaining, the industry continues to focus on the inclusion of barrier systems to reduce said permeation of these compressed gaseous fractions.

Despite these recent industry developments, plastic packages still have not yet made significant inroads into the pressurized product industries where condensable gasses such as hydrocarbons, dimethyl ether, hydrofluorocarbons or fluorocarbons are typically used (e.g., automotive, personal care, or food aerosols). Much less in known, and in fact what is known is sometimes inaccurate, about condensable gas propellants in plastic packages.

For instance, it has been taught that some hydrocarbons (e.g., butane, isobutane, propane) and certain fluorocarbons (CFC11 & CFC12) may be contained in certain types of resins. For example, a reference entitled The Science and Technology of Aerosol Packaging, authored by Michael J. Kakos (John Wiley & Sons, publication 1974 edition, chapter 10) states that nylon, polypropylene, melamine, phenolic and high density polyethylene have all been utilized and then discarded as potential resins for use in producing a plastic aerosol package. Problems that were not able to be overcome included fabrication, permeation, odor, and commercial viability. This reference continues to state that thermoplastics, such as acetal, should be used.

In another reference entitled The Aerosol Handbook, 2^{nd} edition, authored by Monfort A. Johnsen (copyright 1982, chapter 4) it is stated that a plastic resin for use as an aerosol package must demonstrate the following properties: (a) high mechanical strength without brittleness, (b) excellent chemical, creep and permeation resistance, (c) adaptability to production technology [injection molding, injection blow molding, ultrasonic or spin welding, decoration methods], (d) design flexibility and (e) moderate to low cost. Notice this reference's focus on the mechanical properties of the plastic resin. This reference continues to state that “crystalline” OPET (oriented polyethylene teraphthalate having a crystalline chemical structure) has been molded acceptably for use as an aerosol package. The present invention will demonstrate that “crystalline” will not work and that “amorphous” is in fact the proper chemical structure.

Like The Aerosol Handbook reference, The Science and Technology of Aerosol Packaging reference incorrectly reemphasizes the focus on the inherent mechanical strength and permeation characteristics of the plastic resin. More specifically, it states that the primary mechanical concern is creep of the resin over time and temperature. These references continue to suggest that nylon, acetal, polyethylene teraphthalate and polycarbonate are acceptable “engineering resins” for the construction of an aerosol package.

These erroneous concentrations on the mechanical properties of resins, led those skilled in the art to believe that the “polyester family” (e.g., polyethylene teraphthalate, polyethylene naphthalate, polybutylene teraphthalate, etc.) was capable of properly containing hydrocarbon propellants (e.g. butane, isobutane or propane) because they were “strong” enough. The following data helps to support this false or at least incomplete understanding:

Butane Contained Within 100% PET (Mfg: Yoshino):

| | | | Package | Package | Package | |
|---|---|---|---|---|---|---|
| Controlled | | | Swelling | Swelling | Swelling | |
| Temp Room | Time | Discoloring | at Neck | at Mid | at Base | Weight Loss |
| 120 F. | 3 Month | None | 0.26% | 0.00% | 0.11% | 0.63% |
| 77 F. | 3 Month | None | 0.00% | 0.00% | 0.00% | 0.25% |

However, it has been discovered that the mechanical understanding of resins containing hydrocarbons does not translate to other like propellants. For instance, it has been discovered that dimethyl ether (hereinafter referred to as “DME”) is not compatible with polyesters despite the fact that they generate similar internal pressures within a package.

DME Contained Within 100% PET (Mfg: Yoshino):

| | | | Package | Package | Package | |
|---|---|---|---|---|---|---|
| Controlled | | | Swelling | Swelling | Swelling | |
| Temp Room | Time | Discoloring | at Neck | at Mid | at Base | Weight Loss |
| 120 F. | 3 Month | Yes | 40.00% | 8.93% | 26.14% | 100.00% |
| 77 F. | 3 Month | Yes | 14.78% | 4.76% | 8.12% | 47.46% |

Antiperspirant Having 40% DME Contained Within 100% PET (Mfg: Yoshino):

| | | | Package | Package | Package | |
|---|---|---|---|---|---|---|
| Controlled | | | Swelling | Swelling | Swelling | |
| Temp Room | Time | Discoloring | at Neck | at Mid | at Base | Weight Loss |
| 120 F. | 3 Month | Yes | 21.00% | 6.00% | 12.00% | 37.00% |
| 77 F. | 3 Month | Yes | 2.00% | 1.00% | 3.00% | 7.78% |

In fact, dimethyl ether does more than just permeate from the package, it may actually cause the package to change in dimension, change in color or even violently explode.

It should also be recognized that plastic packages containing flammable gasses must undergo additional testing. For example, the Department of Transportation in the United States (referred to as “DOT”) specifies that each package filled with a flammable gas must be subjected to a 130° Fahrenheit environment (referred to as “hot tanking”) prior to shipment in accordance with DOT 49 CFR Ch. 1 [10-1-01], section Research and Special Programs Administration. Another testing requirement example includes the European Counsel Directive 75/324/EEC of May 20, 1975. These additional tests significantly limit plastic functional testing and thus a plastics ability to be brought to market. Additionally, this additional testing worsens the stability of the pressurized plastic package due to increased temperatures that reduce the mechanical strength of the plastic and increase the pressure within the package. While it has been understood that the ambient mechanical strength of a plastic is not alone responsible for robustness, no specific property, parameter or technique until now has been identified to design packages meeting these stringent requirements.

What is needed is an aerosol plastic package capable of containing dimethyl ether and dimethyl ether based products. It has been discovered that an aerosol plastic package having a container body substantially made of, or substantially prepared with an internal layer of, amorphous nylon six [6I/6T] does provide sufficient containment of dimethyl ether and dimethyl ether based products.

### SUMMARY OF THE INVENTION

The present invention provides . . . .

### BRIEF DESCRIPTION OF THE DRAWINGS

While the specification concludes with claims which particularly point out and distinctly claim the present invention, it is believed that the present invention will be better understood from the following description of preferred embodiments, taken in conjunction with the accompanying drawings, in which like reference numerals identify like elements and wherein:

FIG. 1 is a pressurized package of the present invention that is capable of containing a pressurized product; and

FIG. 2 is a pressurized package of the present invention that is capable of containing a pressurized product and has multiple wall layers.

### DETAILED DESCRIPTION OF THE INVENTION

Reference will now be made in detail to various exemplary embodiments of the invention, several of which are also illustrated in the accompanying drawings, wherein like numerals indicate the same elements throughout the views, and numbers with the same final two digits indicate corresponding elements among embodiments.

The term “ambient conditions” as used herein refers to surrounding conditions at about one atmosphere of pressure, at about 50% relative humidity, and at about 25° C.

The term “structurally and chemically compatible” “as used herein refers to a combination of a package and a formula forming a stable product based on their behavior upon storage at 49° C. for 4 weeks and their compliance with special aerosol testing requirements (e.g., DOT 49 CFR Ch. 1 [10-1-01], section Research and Special Programs Administration, and European Counsel Directive 75/324/EEC of May 20, 1975). A stable product will not show visible discoloration or hazing upon said storage or have more than 1.5% weight loss or show more than a 2% change in a given dimension (i.e. diameter, width, depth, length, or crimp height) or rupture or BLEVE.

The term “aerosol package” as used herein means any packaged composition that is pressurized from a gas or liquefied gas propellant, wherein the propellant provides a way for pushing or moving the composition to and/or through an application device. These aerosol products can deliver the composition to its targeted source (e.g., consumers skin, hair, underarm, etc.) in various ways including, but not limited to, a spray or via a porous application surface.

The term “plastic” refers to any synthetic or organic materials that can be molded or shaped, generally when heated, and then hardened into a desired form including, but not limited to, polymers, resins, and cellulose derivatives.

The term “plastic package” refers to the container vessel of the aerosol package being made substantially of plastic. The sealing valve and actuator of the package may or may not necessarily be made substantially of plastic.

All percentages, parts and ratios as used herein are by weight of the total composition, unless otherwise specified. All such weights as they pertain to listed ingredients are based on the active level and, therefore, do not include solvents or by-products that may be included in commercially available materials, unless otherwise specified.

Wishing not to be bound be theory it is believed that one of the governing principles of the present invention is that “likes dissolves likes”. First, this solubility principle is important in determining whether the plastic and the product are compatible. For example, consider the following plastic solubility parameter data:

TABLE 1

| Family of | (δ/Mpa ½) | (δ/cal/cc) |
|---|---|---|
| Resins* | Hildebrand | SI units** |
| Polyfluorocarbon | 12-13 | 6-7 |
| Polyethylene | 16-17 | 8-9 |
| Polyesters | 17-22 | 8-11 |
| Polycarbonate | 19-22 | 9-11 |
| Polyamides | 28-31 | 14-16 |

*It should be noted that within each of the “families” of resins, there may be differences of a unit or more.
**Actual difference between Hildebrand units and SI units is a division by 2.0455

Next, the product solubility parameter should be calculated. For example, which takes into consideration the percentage of each major component and its respective solubility parameter:

TABLE 2

| | (a) | (b) | |
|---|---|---|---|
| | % of | (δ/Mpa ½) | |
| Components | formulation | Hildebrand | = (a) * (b) |
| DME | 40% | 17 | 6.8 |
| Silicones | 30% | 12 | 3.6 |
| Diol/active | 30% | 22 | 6.6 |
| | | | 17.0 |

Lastly, when selecting a resin for making the package to contain the product, one should select a family of resins having a different solubility parameter than the product. Within the current example, the product had a solubility parameter value of 17. Therefore, it is advised that resins other than polyethylene and polyesters be used, such as polyamide. It has been discovered that generally placing a product having a solubility parameter value that is at least +/−5 units different from that of the package solubility parameter value will result in a compatible package-product combination.

Once a suitable resin has been selected to overcome most solubility concerns, other considerations must be made. For instance, it may be desirable to sell the product in a clear (or otherwise transparent) package. It has been discovered that containing DME or a DME-based product in an originally-clear package made of standard polyester caused this package to lose its clarity and/or structural integrity. Therefore, identifying specific types of polyamides for containing DME was essential. Historically, in an effort to make a structurally secure and chemical resistant package, one skilled in the art would use a resin in “crystalline” form. On the contrary, it has been discovered that that amorphous grades of polyamides perform better than the crystalline form of the same chain length (e.g., nylon 6I/6T versus nylon MDX6). While it is currently believed that both forms maybe suitable in various applications, some applications have shown that amorphous 6 carbon chain nylon performs better than amorphous 12 carbon chain nylon. Wishing not to be bound by theory, it has been appreciated that amorphous 12 carbon chain nylon is less hygroscopic and less polar in nature and that these properties may play a role in its use. Even further, it is believed that more polar amorphous nylons perform better than less polar amorphous nylons.

While these discoveries have been found, the theories of why the work are complex. Accordingly, applicants also make mention to the following observed properties of amorphous nylons that may also be important factors when practicing the present invention: high levels of stiffness, high levels of hardness, low tendency to creep, good dimensional stability, little process shrinkage, good heat distortion properties, high melt viscosity, high melt strength, can be easily alloyed with other amorphous or semi-crystalline polyamides, low water uptake, good surface properties, moderate weatherability, and little stress-cracking resistance to polar solvents.

It is also herein contemplated that the present invention may be practiced in many consumer products including, but not limited to, antiperspirants, deodorants, hairsprays, cooking sprays, perfumes, shaving creams/gels, or drug products.

### Manufacturing of Packages

While injection stretch blow molding has proven to be a suitable manufacturer technique, other manufacturing techniques may be used. Various suppliers including, but not limited to, the Owens-Brockway Division of Owens-Illinois are capable of making packages of the present invention (e.g., specification number N-41701).

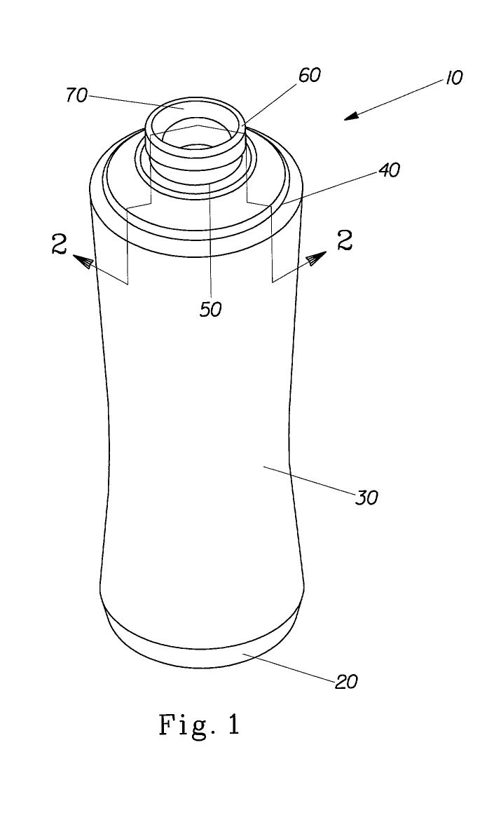

FIG. 1 depicts a non-limiting exemplary embodiment of a suitable package. This package 10 has a minimum wall thickness of 0.045″ with the contacting surface made substantially of amorphous nylon 6I/6T supplied by EMS-CHEMIE under the tradename of Grivory G21. Package 10 has a base 20, a container body 30 for storing (i.e., holding, containing, etc) a product, a shoulder 40 that may include snap or locking features which mate with a closure (not shown), a neck region 50 that supports a valve mechanism (not shown), and a crimping ring 60 that permits valve insertion and adhesion to package 10. Base 20 should be designed to have sufficient thickness to withstand a drop impact (e.g., 6 foot drop test). Container body 30 should be designed to substantially resist deformation under stress. Shoulder 40 and neck region 50 should be designed having sufficient strength to endure the mechanical force exerted during the crimping/clinching process in during manufacturing and product stacking during shipment. Further, it has been discovered that crimping ring 60 should have relief on the internal surface in the form or a recess or chamfer 70 to improve sealing between the valve and container. While package 10 was made having a substantially round cross-section and a fill volume of 2.54 ounces, however, other geometries and fill volumes may be appreciated by one skilled in the art.

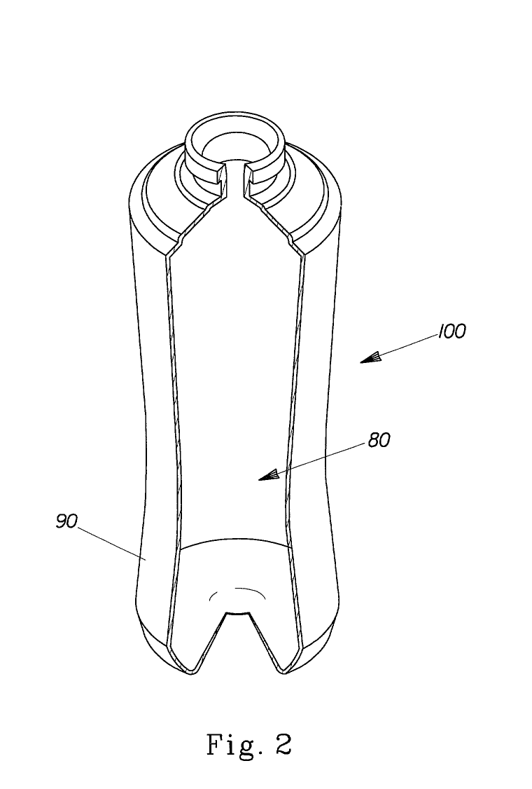

FIG. 2 depicts a non-limiting exemplary embodiment of a suitable package wherein a second layer has been appropriated to provide the necessary barrier properties. Multiple layers may be used to accomplish the intended benefits of the present invention. These multiple layers may be made of the same or different materials. One or more layers may use a cured liquid or a plasma coating to provide the desired barrier properties. In one example, the inner layer 80 of the container body 30 is substantially made of amorphous nylon 6I/6T in order to substantially contain the DME. The outer layer 90 of package 100 is substantially made of any suitable material that is capable of containing the pressurized composition including, but not limited to polyfluorocarbon, polyethylene, polypropylene, polyesters, polycarbonate, polystyrene and polyamides.

### EXAMPLE

Antiperspirant Having 40% DME Contained Within 100% Amorphous Nylon 6.

(Nylon supplier: EMS located in Switzerland; Package manufacturer: Owens-Illinois, located Toledo, Ohio):

| Controlled | | | | | | |
|---|---|---|---|---|---|---|
| Temp | | Dis- | Package | Package | Package | Weight |
| Room | Time | coloring | Neck | Mid | Base | Loss |
| 120 F. | 1 Month | No | 0% | 0% | 0% | 1% |
| 77 F. | 1 Month | No | 0% | 0% | 0% | 0% |

What is claimed is:

1. A pressurized product comprising:
(a) a package, said package being substantially made of a polyamide; and
(b) dimethyl ether, said dimethyl ether is stored within said package, said package and dimethyl ether are structurally and chemically compatible.

2. The pressurized product of claim 1 wherein said polyamide is a nylon.

3. The pressurized product of claim 1 wherein said polyamide is amorphous.

4. The pressurized product of claim 1 wherein said polyamide is an amorphous nylon 6I/6T.

5. The pressurized product of claim 1 wherein said package has a container body made of multiple layers.

6. The pressurized product of claim 6 wherein said multiple layers consists of an inner layer being substantially made of amorphous nylon 6I/6T and an outer layer being substantially made of any material capable of containing a pressurized product.

7. The pressurized product of claim 1 wherein said package is translucent.

8. The pressurized product of claim 1 further comprising a product stored within said package, wherein said product is selected from the group consisting of antiperspirant product, deodorant product, hairsprays, cooking sprays, perfumes, shaving creams/gels, or drug products.

9. A pressurized product comprising:
(a) a package, said package being substantially made of a resin, said resin having a solubility parameter value; and
(b) a pressurized composition, said composition having a solubility parameter value, wherein the resin solubility parameter value being at least +/−5 (δ/Mpa ½) Hildebrand units different from that of the composition solubility parameter value, whereby the package and composition are structurally and chemically compatible.
